(19) United States
(12) Patent Application Publication
(10) Pub. No.: US 2026/0196348 A1
(43) Pub. Date: Jul. 9, 2026

(54) ARTICLES AND METHODS FOR DETECTION OF HIDDEN CARDIOVASCULAR DISEASE FROM PORTABLE ELECTROCARDIOGRAPHIC SIGNAL DATA USING DEEP LEARNING

(71) Applicant: Yale University, New Haven, CT (US)

(72) Inventors: Rohan Khera, New Haven, CT (US); Akshay Khunte, New Haven, CT (US); Veer Sangha, New Haven, CT (US)

(21) Appl. No.: 19/133,767

(22) PCT Filed: Nov. 29, 2023
(86) PCT No.: PCT/US2023/081523

§ 371 Date: May 29, 2025

Related U.S. Application Data
(60) Provisional application No. 63/428,569, filed on Nov. 29, 2022.

(51) Int. Cl. G16H 50/20; A61B 5/00; A61B 5/332

(52) U.S. Cl. CPC G16H 50/20; A61B 5/332; A61B 5/7203

(57) ABSTRACT

Provided herein are methods of training a model to detect cardiovascular disease in a subject from portable electrocardiogram (ECG) signal data and computer-implemented methods of detecting cardiovascular disease in a subject. The methods of training a model includes selecting an ECG dataset corresponding to a cardiovascular disease, the ECG dataset including multiple distinct ECGs, forming a training dataset from the ECG dataset, training the nodes of a deep neural network on the training dataset, and including random gaussian noise with each ECG of the ECG dataset during the training of the nodes. The computer-implemented methods of detecting cardiovascular disease in a subject include applying a deep neural network to ECG data for a subject, the deep neural network being trained according to the training methods disclosed herein.

### CROSS-REFERENCE TO RELATED APPLICATIONS

The present application claims priority under 35 U.S.C. § 119(e) to U.S. Provisional Patent Application No. 63/428,569, filed Nov. 29, 2022, which application is incorporated herein by reference in its entirety.

### BACKGROUND OF THE INVENTION

Advances in wearable and handheld technologies now enable the point-of-care acquisition of single-lead electrocardiogram (ECG) signals, paving the path for efficient and scalable Artificial intelligence (AI) screening tools for use in the wider community. While this improved accessibility could potentially enable broader AI-based screening for structural and functional cardiovascular disease, such as left ventricular systolic dysfunction (LVSD), the reliability of such tools is limited by the presence of noise in data collected from wearable devices. More specifically, unlike wearable-adapted ECG models for arrythmia detection that have demonstrated consistently high sensitivity and specificity, wearable ECG models for LVSD have shown inconsistent performance, which is consistently lower than that observed in the original, clinical development studies. Additionally, although AI-ECG is now recognized as a promising screening tool for LVSD, its use so far has been limited to 12-lead ECGs.

In the absence of large, labelled datasets of wearable ECGs, the development of wearable-adjusted algorithms that can detect underlying structural heart disease relies on single-lead information specifically adapted from 12-lead ECGs extracted from clinical ECG libraries. However, this process does not specifically account for the unique data acquisition challenges encountered with wearable ECG, possibly contributing to their lower diagnostic performance. Indeed, several sources of noise exist in wearable data, arising from factors such as poor electrode contact with the skin, movement and muscle contraction during the ECG, and external electrical interference. This wearable device-specific noise has significant implications, with models demonstrating significantly better performance when tested on high-quality subsets of wearable ECG data instead of all available signals. This marked difference in performance based on noise has limited the scalability of wearable device-based screening programs, with a wearable device-based atrial fibrillation screening study disqualifying 22% of patients due to insufficient signal quality.

Accordingly, there is a need in the art for articles and methods that improve on existing detection methods by accounting for wearable device-specific noise in broadly accessible models that form the basis of effective screening programs. The present invention addresses this need.

### SUMMARY OF THE INVENTION

In one aspect a method of training a model to detect cardiovascular disease in a subject from portable electrocardiogram (ECG) signal data includes selecting an ECG dataset corresponding to a cardiovascular disease, the ECG dataset including multiple distinct ECGs; forming a training dataset from the ECG dataset; training the nodes of a deep neural network on the training dataset; and including random gaussian noise with each ECG of the ECG dataset during the training of the nodes.

In some embodiments, the ECG dataset includes 12-lead ECGs. In some embodiments, forming the training dataset comprises isolating a signal from a single lead of each of the 12-lead ECGs, the isolated signal from each of the 12-lead ECGs forming a single-lead ECG. In some embodiments, the single lead comprises lead I. In some embodiments, forming the training dataset further comprises removing a baseline drift from each of the single-lead ECGs. In some embodiments, forming the training dataset comprises isolating a signal from at least two leads of the 12-lead ECGs, the isolated signals from the at least two leads forming a multi-lead ECG. In some embodiments, the training dataset includes at least two of each ECG in the ECG dataset.

In some embodiments, the random gaussian noise comprises random gaussian noise isolated from one or more distinct frequency ranges. In some embodiments, the random gaussian noise is isolated using at least one of high and low pass filtering. In some embodiments, including the random gaussian noise with each ECG comprises randomly selecting one of the distinct frequency ranges with each of the ECGs and a random small section of the isolated noise. In some embodiments, the frequency range includes at least one of 3-12 Hz, 12-50 Hz, 50-100 Hz, or 100-150 Hz. In some embodiments, including the random gaussian noise with each ECG comprises randomly selecting with each of the ECGs at least one of the distinct frequency ranges and a random small section of isolated noise from one or more of 3-12 Hz, 12-50 Hz, 50-100 Hz, or 100-150 Hz. In some embodiments, the random gaussian noise is included with the ECG at a randomly chosen signal-to-noise ratio of from <1 to >1.

In some embodiments, the model comprises a convolutional neural network (CNN) model. In some embodiments, the cardiovascular disease is left ventricular systolic dysfunction (LVSD).

In another aspect, a computer-implemented method of detecting cardiovascular disease in a subject includes receiving electrocardiogram (ECG) data for the subject from a portable ECG device; applying a deep neural network to the ECG data for the subject, the deep neural network comprising a plurality of nodes trained to distinguish ECG data of a heart with cardiovascular disease from ECG data of a healthy heart according to the method of claim 1; comparing outputs of the nodes to patterns of node outputs for ECG data from healthy subjects and subjects with one or more cardiovascular diseases; and determining if the subject has cardiovascular disease based upon the outputs of the nodes. In some embodiments, the portable ECG device is a wearable ECG device. In some embodiments, the ECG data is single-lead ECG data. In some embodiments, the single-lead ECG data is lead I ECG data.

In a further aspect, an apparatus for detecting cardiovascular disease in a subject from portable electrocardiogram (ECG) signal data includes a processor; a memory unit; and a communication interface. The processor is connected to the memory unit and the communication interface. The processor and memory unit are configured to implement the model trained according to any of the embodiments disclosed herein.

### BRIEF DESCRIPTION OF THE DRAWINGS

FIG. 1 shows graphs illustrating noise extraction and ECG augmentation for noise-adapted training. Abbreviations: ECG, electrocardiogram; Hz, hertz; s, seconds.

FIG. 2 shows graphs illustrating noise augmentation of test set. Abbreviations: ECG, electrocardiogram; s, seconds.

FIG. 3 shows graphs illustrating standard and noise-adapted model performance at increasing levels of noise. Abbreviations: AUROC, area under receiver operating characteristic curve; ECG, electrocardiogram.

FIG. 4 shows images illustrating UMAP projections of last-layer predictions of standard and noise-adapted model for clean and noise-augmented ECGs. Abbreviations: UMAP, uniform manifold approximation and projection; ECG, electrocardiogram.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein are methods of training a model to detect cardiovascular disease in a subject from portable electrocardiogram (ECG) signal data. In some embodiments, the method includes selecting an ECG dataset corresponding to a cardiovascular disease, forming a training dataset from the ECG dataset, and training a set of algorithms on the training dataset through deep learning. The ECG dataset includes multiple ECGs from subjects with and without a cardiovascular disease of interest.

In some embodiments, the ECG dataset includes clinical device 12-lead ECG signals. These 12-lead ECG signals may be processed during the formation of the training dataset to isolate one or more signals for detection of cardiovascular disease from single- or multi-lead ECG signals, such as those provided by many portable ECG devices. For example, in some embodiments, forming the training dataset includes isolating a signal from a single lead (e.g., lead I) of each 12-lead ECG in the ECG dataset, the isolated signal from each of the 12-lead ECGs forming a single-lead ECG. The single-lead ECG signal may be isolated by any suitable method, such as, but not limited to, preprocessing with median pass filtering and scaling to millivolts. After isolating the single-lead ECG, baseline drift may be removed by calculating a one second median filter and subtracting it from the single-lead ECG. As will be appreciated by those skilled in the art, the disclosure is not limited to a single lead or lead I, and may include any other suitable lead or combination of leads (multi-lead).

During the training of the set of algorithms, random gaussian noise is included with each ECG in the training dataset. In some embodiments, the random gaussian noise is isolated from within one or more distinct frequency ranges. In some embodiments, isolating the distinct frequency ranges includes applying high pass and low pass filters to a recording of random gaussian noise (e.g., five minutes of random gaussian noise). The frequency ranges include any suitable range based upon the noise in the portable ECG signal. In some embodiments, the gaussian noise is isolated from within one or more ranges that model an element of real-world ECG noises. For example, the frequency ranges may include 3-12 Hz, 12-50 Hz, 50-100 Hz, and 100-150 Hz, where the 3-12 Hz range models the motion artifact noises attributable to tremors, the 50-100 Hz domain reflects consistent electrode contact noise, and the 12-50 Hz and 100-150 Hz ranges contain the lower and higher frequency muscle noises, respectively. Additionally, the 50-100 and 100-150 Hz ranges each contain multiples of 50 and 60 Hz, the two mains frequencies used in ECG acquisition, which make up powerline interference noise.

Once isolated into distinct frequency ranges, one range is independently and randomly selected for inclusion with each ECG during the training. In some embodiments, each ECG is included more than once in the training dataset. For example, in some embodiments, the training dataset includes two of each single-lead ECG, and random gaussian noise from one of the four distinct frequency ranges is randomly included with each of the single-lead ECG during training.

Additionally, in some embodiments, a set sequence length (e.g., ten seconds) is randomly selected from the full random gaussian noise recording and then added to the clean ECG at a signal-to-noise ratio (SNR) randomly selected from a set of SNRs, including 0.50, 0.75, 1.00, and 1.25.

In some embodiments, the training with the noise-adapted model includes randomly subsetting all unique patients represented in the set of ECGs into training, validation, and held-out test sets (e.g., 85%, 5%, 10%). The noise-adapted model is then trained on a neural network or another deep learning-based approach at a desired learning rate until a set performance level is reached. For example, in some embodiments, the noise-adapted model is trained at a learning rate of 0.001 for one epoch, then at a learning rate of 0.0001 until performance on the validation set did not improve for three consecutive epochs. The epoch with the highest performance on the validation set may be selected for each model. In some embodiments, the deep neural network includes a plurality of nodes trained to distinguish a noise-augmented ECG recording of a heart with one or more cardiovascular diseases from a noise-augmented ECG recording of a healthy heart. In such embodiments, the method also includes comparing outputs of the nodes from the ECG reading for the subject to patterns of node outputs for ECG readings of healthy subjects and subjects with one or more cardiovascular diseases. The determining step is then based upon the comparison of the outputs of the nodes.

Although described herein primarily with respect to a deep neural network including a plurality of nodes, as will be appreciated by those skilled in the art, the disclosure is not so limited and may include any other suitable machine learning-based method. For example, in some embodiments, the neural network includes a convolutional neural network (CNN) coupled to a fully connected network, or a transformed-based method. In embodiments that use a CNN, the CNN includes any suitable number and/or size of convolutional layers and total model parameters. In some embodiments, the CNN includes the architecture yielding the highest area under the receiver operator characteristic (AUROC) curve on the validation set with the fewest number of parameters for the standard model. For example, one such architecture may include a (5000, 1, 1) input layer, corresponding to a 10-second, 500 Hz, Lead I ECG, followed by seven two-dimensional convolutional layers, each of which is followed by a batch normalization layer,

ReLU activation layer, and a two-dimensional max-pooling layer. The output of the seventh convolutional layer is taken as input into the fully connected network, which includes two dense layers. Each of the dense layers is followed by a batch normalization layer, ReLU activation layer, and a dropout layer with a dropout rate of 0.5. The output layer includes a dense layer with one class and a sigmoid activation function. Additionally or alternatively, in some embodiments, model weights are calculated for the loss function such that learning is not affected by a lower frequency of subjects without a condition (e.g., LVEF<40%) compared to the incidence of subjects with the condition (e.g., LVEF≥40%) using the effective number of samples class re-weighting scheme.

The inclusion of the random gaussian noise during the training forms a noise-adapted model that provides significantly improved performance on ECGs augmented with real-world noises, such as those isolated from portable device ECGs, as compared to existing methods that rely upon ECG signal data collected from hospital devices with less noise. More specifically, by explicitly making the algorithms learn ECG features on artificially noised ECGs at the same time as they learn to detect hidden features of structural and functional heart disease that are not discernable to humans, the methods disclosed herein train the algorithms to process noisy portable ECGs signals without relying on datasets with raw wearable device ECG signal data, which are not widely available. Accordingly, the training through deep learning according to the embodiments disclosed herein creates a set of algorithms capable of detecting structural and functional abnormalities of the heart from any noisy wearable single or multi-lead ECG signal with consistent performance, regardless of the type, frequency, and amplitude of the noise.

Also provided herein are computer-implemented methods of detecting cardiovascular disease in a subject. In some embodiments, the method includes receiving ECG data for a subject from a portable ECG device; applying a deep neural network to the ECG data for the subject, the deep neural network comprising a plurality of nodes trained to distinguish ECG data of a heart with cardiovascular disease from ECG data of a healthy heart according to one or more of the training methods disclosed herein; comparing outputs of the nodes to patterns of node outputs for ECG data from healthy subjects and subjects with one or more cardiovascular diseases; and determining if the subject has cardiovascular disease based upon the outputs of the nodes. The ECG data includes any suitable data, such as, but not limited to, single-lead ECG data, multi-lead ECG data, or any other suitable data. Suitable portable ECG devices include, but are not limited to, a wearable ECG device, single-lead ECG devices, multi-lead ECG devices, or any other suitable device.

Suitable cardiovascular diseases for detection with the presently disclosed methods include, but are not limited to, structural disorders of the heart and/or structures supporting the heart, functional disorders of the heart and/or structures supporting the heart, or a combination thereof. Such disorders may arise from abnormalities of the muscle, valves, blood vessels, and/or the lining of the heart, and may be due to genetic causes, environmental causes, lifestyle causes, unknown precipitants of the disease, or combinations thereof. For example, in some embodiments, the disease includes low ejection fraction (EF) of the left ventricle (LVEF), where low EF includes any EF of less than 40%. In such embodiments, the ECG dataset includes a subset with normal EF (i.e., normal subset) and a subset with low EF (i.e., diseased subset). Other suitable diseases include, but are not limited to, left or right ventricular systolic dysfunction (LVSD), left ventricular diastolic dysfunction, right-sided heart failure, aortic and mitral valve disease, including their stenosis or regurgitation, cardiomyopathy and its various subtypes, pulmonary hypertension, as well as other rare genetic cardiac disorders.

In some embodiments, the cardiovascular disease includes a disease that is not normally discernable by physicians from ECG data. For example, in some embodiments, the deep neural network detects a cardiovascular disease present in a patient at the time of the ECG reading. In some embodiments, the method includes identifying hidden clinical labels in ECG data that are associated with a disease. Additionally, or alternatively, in some embodiments, the methods disclosed herein detect underlying cardiovascular disorders and/or predict their future risk.

As discussed in detail above, the methods disclosed herein provide the ability to screen and diagnose structural and functional heart diseases using ECG signals obtained from wearable devices with a model trained on clinical device ECG signals. The noise-resilience allows the algorithms to retain performance irrespective of the device or context in which the ECG is acquired. Additionally, the algorithms are automated and do not require human input in data extraction. Accordingly, the methods disclosed herein facilitate use directly by end users, such as patients and clinicians, on data extracted from patients' personal wearable devices. Since wearable devices are a significantly more accessible and cost-effective means of acquiring an ECG compared to a hospital ECG device, the methods disclosed herein also allow for the widespread usage of the algorithms. This widespread usage and accessibility further facilitates early diagnosis and identification of disease and/or future risk, which can prevent morbidity, premature mortality, and lost productivity.

Also provided herein is a computer device including a processor, a memory unit, and a communication interface. The processor is connected to the memory unit and the communication interface. In some embodiments, the processor and memory unit are configured to implement the method according to any of the embodiments disclosed herein. In some embodiments, the processor and memory unit are configured to implement the algorithms trained according to any of the embodiments disclosed herein.

Further provided herein is a computer readable storage medium storing computer-executable instructions for performing the method and/or implementing the algorithms trained according to any of the embodiments disclosed herein.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

### Introduction

Artificial intelligence (AI) can detect left ventricular systolic dysfunction (LVSD) from electrocardiograms (ECGs), a diagnosis that has traditionally relied on comprehensive echocardiography. Even though AI-ECG is now recognized as a promising screening tool for LVSD, its use so far has been limited to 12-lead ECGs. Advances in wearable and handheld technologies now enable the point-of-care acquisition of single-lead ECG signals, paving the path for efficient and scalable AI screening tools for use in the wider community. Whereas this improved accessibility could enable broader AI-based screening for LVSD, the reliability of such tools is limited by the presence of noise in data collected from wearable devices. Unlike wearable-adapted ECG models for arrythmia detection that have demonstrated consistently high sensitivity and specificity, wearable ECG models for LVSD have shown inconsistent performance, which is consistently lower than that observed in the original, clinical development studies.

In the absence of large, labelled datasets of wearable ECGs, the development of wearable-adjusted algorithms that can detect underlying structural heart disease relies on single-lead information specifically adapted from 12-lead ECGs extracted from clinical ECG libraries. However, this process does not specifically account for the unique data acquisition challenges encountered with wearable ECG, possibly contributing to their lower diagnostic performance. Indeed, several sources of noise exist in wearable data, arising from factors such as poor electrode contact with the skin, movement and muscle contraction during the ECG, and external electrical interference. This wearable device-specific noise has significant implications, with models demonstrating significantly better performance when tested on high-quality subsets of wearable ECG data instead of all available signals. This marked difference in performance based on noise has limited the scalability of wearable device-based screening programs, with a wearable device-based atrial fibrillation screening study disqualifying 22% of patients due to insufficient signal quality. Accounting for this noise is a prerequisite to develop broadly accessible models that will form the basis of effective screening programs for LVSD in the community.

In the present study, we hypothesized that a novel, noise-enhanced training approach can boost the performance of wearable-adapted, single-lead ECG models for accurate and noise-agnostic identification of LVSD. Our method, which relies on noise-enhanced training of single-lead ECG data directly derived from clinical ECGs paired with corresponding echocardiograms, explicitly accounts for noise patterns observed in wearable devices.

### Methods

### Study Design

The study was designed as a retrospective analysis of a cohort of 116,210 patients, 18 years of age or older, who underwent clinically indicated ECG with paired echocardiograms within 15 days of the index ECG at the Yale-New Haven Health hospital. To ensure the generalizability of our models, we applied no exclusion criteria, including patients of all sexes, races, and ethnicities.

### Data Source and Population

Raw voltage data for lead I was isolated from 12-lead ECGs collected at the Yale-New Haven Hospital (YNHH) between 2015 and 2021. Lead I was chosen as it represents the standard lead obtained from wearable devices. Each clinical ECG was recorded as a standard 10-second, 12-lead recording with a sampling frequency of 500 Hz. Most ECGs were recorded using Philips PageWriter and GE MAC machines. Patient identifiers were used to link ECGs with an accompanying transthoracic echocardiogram within 15 days of the ECG. These echocardiograms had been evaluated by expert cardiologists, and the LVEF defined in their interpretation was identified. If multiple echocardiograms were performed within the 15-day window, the one nearest to each ECG was used to define the LVEF for the model development and evaluation.

### Data Preprocessing

A standard preprocessing strategy was used to isolate signal from lead I of 12-lead ECGs, that included median pass filtering and scaling to millivolts. The Lead I signal was then isolated from each ECG, and a one second median filter was calculated for and subtracted from each single-lead ECG to remove baseline drift. The amplitudes of each sample in each recording were then divided by a factor of 1000 to scale the voltage recordings to millivolts.

### Isolation of Frequency-Banded Gaussian Noise

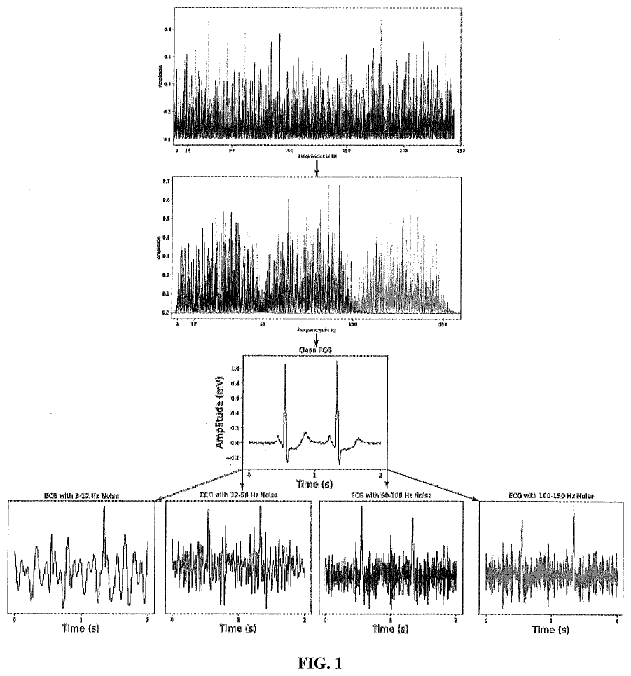

Random gaussian noise within four distinct frequency ranges was isolated to train the noise-adapted model. High pass and low pass filters were applied to five minutes of random gaussian noise to isolate noise within each of the frequency ranges, which included 3-12 Hz, 12-50 Hz, 50-100 Hz, and 100-150 Hz. Each frequency range was specifically selected to model an element of real-world ECG noises. 3-12 Hz models the motion artifact noises attributable to tremors, which occur within this frequency range. The 50-100 Hz domain reflects consistent electrode contact noise, while the 12-50 Hz and 100-150 Hz ranges contain the lower and higher frequency muscle noises, respectively. Additionally, the 50-100 and 100-150 Hz ranges each contain multiples of 50 and 60 Hz, the two mains frequencies used in ECG acquisition, which make up powerline interference noise. (FIG. 1)

### Acquisition of Real-World, Public ECG Noise Recordings

Four real-world noise records, not included during training of either model, were used to test both models. These included three half-hour noise recordings from the MIT-BIH Noise Stress Test Database. The MIT-BIH dataset noises, each obtained at a sampling frequency of 360 Hz, represent three types of noises frequently encountered in ECGs: baseline wander noise, a low-frequency noise produced by lead or subject movement, muscle artifact, caused by muscle contractions, and electrode motion artifact, which is caused by irregular movement of the electrodes during ECG recordings. Each of these recordings were obtained using a standard 12-lead ECG recorder by positioning the electrodes on patient limbs such that the patients' ECG signals were not visible in the recordings.

Noise Extraction from a Portable Device ECG

Real-world noise was also isolated from a 30-second, 300 Hz portable device ECG recording obtained using a KardiaMobile 6L portable ECG device. The noise was extracted from the recording by applying a modified version of the Fourier transform-based approach previously used to denoise ECGs.¹ First, a fast Fourier transform (FFT) was applied to a 30-second ECG recording and the result was plotted in the frequency domain. Then, a threshold was manually selected to separate the high- and low-amplitude frequencies which contained signal and noise, respectively. Finally, instead of computing the inverse FFT on the frequencies with amplitudes greater than this threshold, an inverse FFT was applied to all frequencies with amplitudes below the selected threshold, yielding the noise.

### Noise Augmentation

While training the noise-adapted model, each ECG was included in the training dataset twice, with one of the four frequency ranges for the generated noise randomly selected each time an ECG was loaded. A 10-second sequence was then randomly selected from the full five-minute length of the selected noise recording. This noise sequence was then added to the clean ECG at a signal-to-noise ratio (SNR) randomly selected from a set of SNRs, including 0.50, 0.75, 1.00, and 1.25.

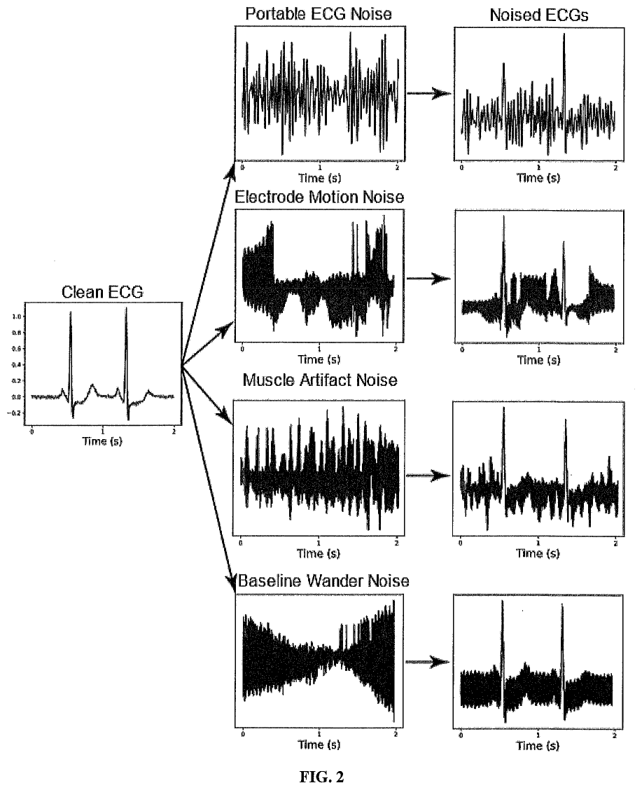

During model evaluation, the noised versions of the test set were generated by following the same procedure as with the noised training set with several key modifications. First, the noise added to ECGs in the test set was sourced from either the baseline wander, electrode motion, or muscle artifacts noise recordings from the MIT-BIH dataset or from the 30-second noise sample from the KardiaMobile 6L portable ECG device. Second, the MIT-BIH noises and the portable ECG noise were all up-sampled from their original sampling frequencies, 360 Hz and 300 Hz, respectively, to a 500 Hz sampling frequency to match that of the clinical device ECGs. Third, the specific 10-second sequence of noise added to each ECG in the test set was randomly selected once and defined for each ECG, ensuring that every time any model was tested at any SNR for any specific noise, each individual ECG was always loaded with the same randomly selected sequence of noise. Model performance was evaluated separately for a larger set of SNRs, including all the SNRs used in training and SNRs of 1.50, 1.75, and 2.00 (FIG. 2).

### Outcome Label

Each ECG included in the dataset had a corresponding LVEF value from a paired echocardiogram within 15 days of the ECG. The cutoff for low LVEF was set as LVEF<40%, a threshold present in most heart failure diagnosis guidelines, and consistent with prior work in this space.

### Model Training

All unique patients represented in the set of ECGs were then randomly subset on the patient level into training, validation, and held-out test sets (85%, 5%, 10%). We built and tested multiple convolutional neural network (CNN) models with varying numbers and sizes of convolutional layers and total model parameters. We selected the architecture yielding the highest area under the receiver operator characteristic (AUROC) curve on the validation set with the fewest number of parameters for the standard model. This architecture consisted of a (5000, 1, 1) input layer, corresponding to a 10-second, 500 Hz, Lead I ECG, followed by seven two-dimensional convolutional layers, each of which were followed by a batch normalization layer, ReLU activation layer, and a two-dimensional max-pooling layer. The output of the seventh convolutional layer was then taken as input into a fully connected network consisting of two dense layers, each of which were followed by a batch normalization layer, ReLU activation layer, and a dropout layer with a dropout rate of 0.5. The output layer was a dense layer with one class and a sigmoid activation function. Model weights were calculated for the loss function such that learning was not affected by the lower frequency of LVEF<40% compared to the incidence of LVEF≥40% using the effective number of samples class re-weighting scheme.²

Both models were trained on the Keras framework in TensorFlow 2.9.1 and Python 3.9 using the Adam optimizer. First, the models were trained at a learning rate of 0.001 for one epoch. The learning rate was then lowered to 0.0001 and training was continued until performance on the validation set did not improve for three consecutive epochs. The epoch with the highest performance on the validation set was selected for each model.

### Learning Representation Assessments for Noise-Adapted Model

To visualize the variation between predictions for clean and noise-augmented data for each model, we first modified both the standard and noise-adapted models by removing their final output layers so both models instead produced the 320-dimensional vector output of the model's final fully connected layer. We then randomly selected a 1,000 ECG subset from the held-out test set. For each of the two models, we generated the 320-dimension prediction vectors five times for each of the 1,000 ECGs—once without noise, and once augmented at an SNR of 0.5 for each of the four noises used for testing. We then visualized the variation in the predictions separately for the standard and noise-adapted models using uniform manifold approximation and projection (UMAP), which constructs a two-dimensional representation of the 320-dimension prediction vectors.³ The variation in predictions was numerically assessed by the pair-wise calculation of the Euclidean distances between the 320-dimensional prediction vectors for the clean and noised data for each of the four noises. These Euclidean distances were then scaled on a per-model and per-noise basis by dividing by the total range of pair-wise Euclidean distances for each model and noise combination. The average scaled Euclidean distance and a 95% confidence interval was then calculated for each model and noise combination.

### Statistical Analysis

Summary statistics are presented as counts (percentages) and median (interquartile range, IQR), for categorical and continuous variables, respectively. Model performance was evaluated in the held-out test set both with and without added real-world noises. We used area under receiving operation characteristics (AUROC) to measure model discrimination. We also assessed area under precision recall curve (AUPRC), sensitivity, specificity, positive predictive value (PPV), negative predictive value (NPV), and diagnostic odds ratio, and chose threshold values based on cutoffs that achieved sensitivity of 0.90 in validation data. All analyses were performed using Python 3.9 and level of significance was set at an alpha of 0.05.

### Results

### Study Population

There were 2,135,846 consecutive 12-lead ECGs collected by YNHH between 2015 and 2021, 440,072 of which had accompanying TTEs acquired within 15 days of the ECG. We developed the model on 385,601 of the ECG-TTE pairs, representing 116,210 unique patients, which contained a full 10 seconds of uninterrupted recordings across all 12 leads. The Lead I signal was then isolated from each 12-lead ECG. All selected single-lead ECGs contained 10 seconds of Lead I signal at a 500 Hz sampling rate. The single-lead ECGs were then split at a patient level into train, validation, and test datasets.

Patients had a median age of 68 years (IQR 56, 78) at the time of ECG recording and 50,776 (43.7%) of the patients were women. 75,928 (65.3%) of patients were non-Hispanic white individuals, 14,000 (12.0%) were non-Hispanic Black individuals, 9,349 (8.0%) were Hispanic individuals, and 16,843 (14.5%) were from other racial backgrounds. For LVEF, 56,894 (14.8%) of recordings had an LVEF below 40%, 40,240 (10.4%) had an LVEF between 40% and 50%, and the remaining 288,467 (74.8%) had an LVEF of 50% or higher.

### Detection of LV Systolic Dysfunction

The noise-adapted and standard models performed similarly on the held-out test set without added noise, with an AUROC for detection of LVEF<40% of 0.90 (95% CI 0.89-0.91) and 0.90 (95% CI 0.88-0.91), respectively. AUPRC on this clean held-out test set was 0.46 and 0.48, respectively. The noise-adapted model had specificity and sensitivity of 0.68 and 0.92, respectively and a PPV and NPV of 0.20 and 0.99, respectively. The standard model had sensitivity and specificity of 0.69 and 0.91, respectively and a PPV and NPV of 0.21 and 0.99, respectively. The noise-adapted model's performance was comparable across subgroups of age, sex, and race (Table 1).

TABLE 1
Performance of noise-adapted model on test ECGs
without noise across demographic subgroups.

| Labels | PPV | NPV | Specificity | Sensitivity | AUROC (95% CI) | AUPRC | OR |
|---|---|---|---|---|---|---|---|
| All | 0.203 | 0.990 | 0.676 | 0.924 | 0.896 (0.886-0.905) | 0.455 | 25.195 |
| Male | 0.231 | 0.986 | 0.653 | 0.916 | 0.881 (0.867-0.894) | 0.478 | 20.508 |
| Female | 0.163 | 0.994 | 0.716 | 0.923 | 0.913 (0.898-0.927) | 0.404 | 30.318 |
| White | 0.204 | 0.990 | 0.675 | 0.922 | 0.894 (0.883-0.906) | 0.445 | 24.544 |
| Black | 0.211 | 0.990 | 0.596 | 0.945 | 0.880 (0.854-0.906) | 0.461 | 25.407 |
| Hispanic | 0.208 | 0.992 | 0.740 | 0.923 | 0.917 (0.881-0.953) | 0.535 | 34.158 |
| Other | 0.218 | 0.995 | 0.772 | 0.941 | 0.932 (0.900-0.964) | 0.545 | 54.122 |
| 65 or Older | 0.198 | 0.989 | 0.606 | 0.936 | 0.879 (0.865-0.893) | 0.432 | 22.611 |
| Under 65 | 0.209 | 0.990 | 0.731 | 0.910 | 0.908 (0.895-0.921) | 0.481 | 27.543 |

Abbreviations: PPV, positive predictive value; NPV, negative predictive value; AUROC, area under receiver operating characteristic curve; CI: confidence interval, AUPRC, area under precision recall curve; OR, odds ratio.

### Comparison of Standard and Noise-Adapted Model Performance on Noised ECGs

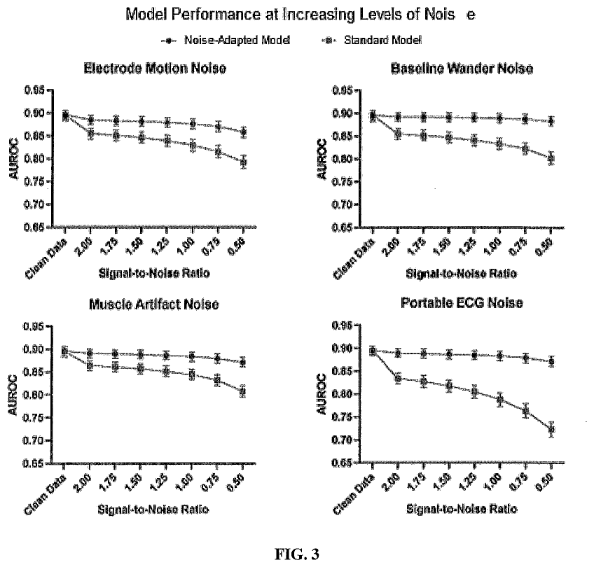

For the noise-adapted model, model performance was comparable across all SNRs for each noise with AUROC between 0.87-0.89, 0.86-0.89, 0.87-0.89, and 0.88-0.89 for portable ECG, electrode motion, muscle artifact, and baseline wander noise, respectively. The standard model had lower performance across all SNRs for every noise, with AUROC between 0.72-0.83, 0.79-0.86, 0.81-0.86, and 0.80-0.86 for portable ECG, electrode motion, muscle artifact, and baseline wander noise, respectively. The difference in performance between the two models was most pronounced with the ECGs augmented with portable ECG noise at an SNR of 0.5, with an AUROC of 0.87 (95% CI 0.86-0.88) and 0.72 (95% CI 0.71-0.74) for the noise-adapted and standard models, respectively. (Table 2, FIG. 3).

TABLE 2
Performance of noise-adapted and standard model on test ECGs with across different types of noise.

| Model | Noise | SNR | PPV | NPV | Specificity | Sensitivity | AUROC (95% CI) | AUPRC |
|---|---|---|---|---|---|---|---|---|
| Noise-Adapted | Clean | N/A | 0.203 | 0.990 | 0.676 | 0.924 | 0.896 (0.886-0.905) | 0.455 |
| | Portable ECG | 0.5 | 0.152 | 0.993 | 0.523 | 0.956 | 0.871 (0.861-0.882) | 0.392 |
| | Portable ECG | 2 | 0.188 | 0.992 | 0.636 | 0.940 | 0.889 (0.880-0.899) | 0.439 |
| | Electrode Motion | 0.5 | 0.126 | 0.993 | 0.395 | 0.971 | 0.858 (0.846-0.869) | 0.361 |
| | Electrode Motion | 2 | 0.167 | 0.991 | 0.579 | 0.945 | 0.885 (0.875-0.895) | 0.426 |
| Standard | Muscle Artifact | 0.5 | 0.175 | 0.989 | 0.608 | 0.927 | 0.871 (0.861-0.882) | 0.389 |
| | Muscle Artifact | 2 | 0.194 | 0.990 | 0.654 | 0.930 | 0.891 (0.881-0.900) | 0.449 |
| | Baseline Wander | 0.5 | 0.186 | 0.990 | 0.634 | 0.932 | 0.883 (0.873-0.893) | 0.427 |
| | Baseline Wander | 2 | 0.201 | 0.991 | 0.669 | 0.929 | 0.892 (0.883-0.902) | 0.457 |
| | Clean | N/A | 0.207 | 0.988 | 0.688 | 0.910 | 0.895 (0.884-0.905) | 0.475 |
| | Portable ECG | 0.5 | 0.091 | 0.981 | 0.132 | 0.972 | 0.723 (0.706-0.739) | 0.200 |
| | Portable ECG | 2 | 0.125 | 0.991 | 0.398 | 0.958 | 0.834 (0.822-0.847) | 0.329 |
| | Electrode Motion | 0.5 | 0.088 | 0.988 | 0.079 | 0.990 | 0.792 (0.779-0.806) | 0.239 |
| | Electrode Motion | 2 | 0.152 | 0.988 | 0.537 | 0.925 | 0.855 (0.843-0.866) | 0.333 |
| | Muscle Artifact | 0.5 | 0.100 | 0.991 | 0.210 | 0.979 | 0.807 (0.795-0.820) | 0.245 |
| | Muscle Artifact | 2 | 0.181 | 0.988 | 0.631 | 0.912 | 0.864 (0.853-0.875) | 0.344 |
| | Baseline Wander | 0.5 | 0.095 | 0.993 | 0.158 | 0.987 | 0.802 (0.789-0.816) | 0.236 |
| | Baseline Wander | 2 | 0.169 | 0.986 | 0.599 | 0.908 | 0.855 (0.843-0.866) | 0.322 |

Abbreviations: SNR, signal-to-noise ratio, PPV, positive predictive value; NPV, negative predictive value; AUROC, area under receiver operating characteristic curve; CI: confidence interval, AUPRC, area under precision recall curve.

### Visualization of Variation in Standard and Noise-Adapted Predictions for Clean and Noise-Augmented ECGs

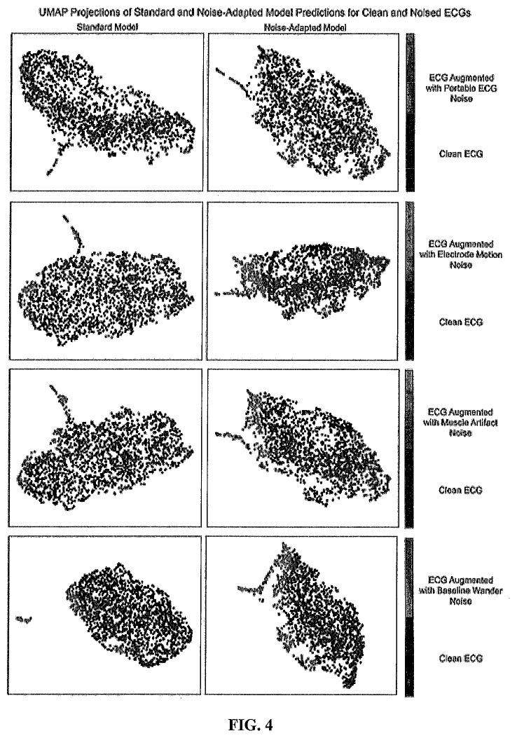

Each UMAP projection of the output of the standard model's final fully connected layer shows separation between the predictions for noised ECGs and predictions for clean ECGs, despite the predictions being for the same set of 1,000 ECGs, with and without each type of noise. This separation is not as evident in the predictions of the noise-adapted model, indicating that the predictions of the noise-adapted model are not shifted to the same degree as the standard model due to the addition of noise (FIG. 4). The scaled Euclidean distances between predictions for noised and clean versions of the ECGs were lower across all four noises. The average scaled Euclidean distances ranged from 0.36 (95% CI: 0.36-0.37) for baseline wander noise to 0.49 (95% CI: 0.48-0.50) for electrode motion noise, respectively for the noise-adapted model. For the standard model, the average scaled Euclidean distances ranged from 0.50 (95% CI: 0.49-0.51) for portable ECG noise to 0.56 (95% CI: 0.55-0.58) for electrode motion noise, respectively (Table 3).

TABLE 3
Scaled Euclidean distance between last-layer predictions of
noise-adapted and standard models for noise-augmented and
clean ECGs across different noise types at an SNR of 0.5.

| Model | Noise | Scaled Euclidean Distance (95% CI) |
|---|---|---|
| Noise-Adapted | Portable ECG | 0.41 (0.40-0.42) |
| | Electrode Motion | 0.49 (0.48-0.50) |
| | Muscle Artifact | 0.40 (0.39-0.41) |
| | Baseline Wander | 0.36 (0.36-0.37) |
| Standard | Portable ECG | 0.50 (0.49-0.51) |
| | Electrode Motion | 0.56 (0.55-0.58) |
| | Muscle Artifact | 0.53 (0.52-0.54) |
| | Baseline Wander | 0.52 (0.51-0.53) |

Abbreviations: SNR, signal-to-noise ratio; ECG, electrocardiogram; CI, confidence interval.

### Discussion

We developed a noise-adapted method of training a deep-learning algorithm that accurately identifies LV systolic dysfunction from single-lead ECG data. This noise-adapted model generalizes significantly better to noise-augmented ECGs than a standard model, enabling greater performance retention for models deployed on ECGs derived from wearable devices. The algorithm demonstrates excellent discriminatory performance even on ECGs containing twice as much noise as signal, features that make it ideal for wearable device-based screening strategies. Notably, the algorithm was developed and validated in a diverse population and demonstrates consistent performance across subgroups of age, sex, and race. From a clinical standpoint, this noise-adapted approach has the potential to expand the screening of LV systolic dysfunction to low-resource settings with limited access to hospital-grade equipment. However, on a more general note, it also defines a novel paradigm on how to build robust, wearable-adapted, single-lead ECG cardiovascular screening models.

Noise-adapted training of deep learning algorithms represents a relatively novel field of AI research, focused on expanding the use of AI tools to everyday life by accounting for noise and artifacts that may preclude their reliable use in this setting. Models trained on clinical ECGs have been applied to wearable device ECGs, but have traditionally shown significantly lower performance on wearable device ECGs than on held-out clinical ECG test sets. Due to the lack of publicly available wearable device ECG datasets, however, development of models for direct use with wearable ECG data is not currently feasible. Furthermore, current clinical ECG signal-based models are limited by the health system wide investment necessary to incorporate them into the clinical workflow, something that may not be available or cost-effective for smaller hospitals and clinics. Wearable device ECGs are significantly more accessible and allow for community-wide screening, an important next step in the early detection of common and rare cardiomyopathies. On this note, our approach represents a major advancement both from a methodological and a clinical standpoint. First, it augments clinical ECG datasets in such way that it enables reliable modelling of noisy, wearable-derived, single-lead ECG signals. Second, it demonstrates that through noise-adjusted single-lead ECG models can retain the prognostic performance of 12-lead ECG models, as shown here for the task of predicting LV systolic dysfunction.

The projection of the predictions of the final non-output layer of the two models using UMAP assists in visualizing the difference between the standard and the noise-adapted model. Compared to the standard model, the noise-adapted model resulted in a significantly greater degree of overlap between the predictions for the noised and original versions of the same ECG signal, highlighting the model's robustness in discerning signal from noise. Furthermore, the noise-adapted model demonstrates significantly less variation in its predictions even when there is twice as much noise as signal in the noise augmented ECGs. Given that none of the noise types used for model testing were included in the training set, this indicates that the noise-adapted model successfully denoises ECG signals prior to generating predictions even for unseen noise. This is particularly important for a model intended for use on wearable devices, which capture ECGs in unpredictable settings with varying types and magnitudes of noise.

This study has certain limitations. First, this model was developed using ECGs from patients who had both an ECG and a clinically indicated echocardiogram. Though this population differs from the intended broader real-world use of this algorithm as a screening method for LV systolic dysfunction among individuals with no clinical disease, the consistent performance across demographic subgroups suggests robustness and generalizability of the model's performance. Nevertheless, prospective assessments in the intended screening setting are warranted. Second, the model performance may vary by the severity of LV systolic dysfunction. Though the LVEF threshold of 40% was selected due to its therapeutic implications, it is possible that the model performance among patients with an LVEF near to this cutoff differed compared to those individuals with LVEF significantly higher or lower than 40%. This might also be attributable to a lack of precision in LVEF measurement by echocardiography, which has shown to be less precise relative to other approaches, such as magnetic resonance imaging. Finally, four distinct types of randomly generated noise were used during training and randomly selected sequences of four real-world noises were used at multiple signal-to-noise ratios in the evaluation of performance on the held-out test set. Though this suggests that the model performance generalizes well to unseen noise, we cannot ascertain whether it maintains performance on every type and magnitude of noise possible on wearable devices.

### Conclusions

We developed a wearable-adapted AI-ECG model for the detection of LV systolic dysfunction which generalizes better than traditional approaches to noisy, single-lead ECGs. Our approach defines a roadmap on how to develop noise-adapted, robust screening tools for use with wearable devices. Given the accessibility and ease-of-use of modern wearable devices, the proposed technology has the potential to expand the screening of LV systolic dysfunction, as well as other cardiovascular conditions, across diverse populations and in resource-limited settings.

The disclosures of each and every patent, patent application, and publication cited herein are hereby incorporated herein by reference in their entirety.

### REFERENCES

1. Kumar, A., Ranganatham, R., Komaragiri, R. & Kumar, M. Efficient QRS complex detection algorithm based on Fast Fourier Transform. Biomed Eng Lett 9, 145-151 (2019).
2. Cui, Jia, Lin & Song. Class-balanced loss based on effective number of samples. Proc. Estonian Acad. Sci. Biol. Ecol.
3. McInnes, L., Healy, J. & Melville, J. UMAP: Uniform Manifold Approximation and Projection for Dimension Reduction. arXiv e-prints arXiv:1802.03426 (2018).

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of training a model to detect cardiovascular disease in a subject from portable electrocardiogram (ECG) signal data, the method comprising:
selecting an ECG dataset corresponding to a cardiovascular disease, the ECG dataset including multiple distinct ECGs;
forming a training dataset from the ECG dataset;
training the nodes of a deep neural network on the training dataset; and
including random gaussian noise with each ECG of the ECG dataset during the training of the nodes.

2. The method of claim 1, wherein the ECG dataset includes 12-lead ECGs.

3. The method of claim 2, wherein forming the training dataset comprises isolating a signal from a single lead of each of the 12-lead ECGs, the isolated signal from each of the 12-lead ECGs forming a single-lead ECG.

4. The method of claim 3, wherein the single lead comprises lead I.

5. The method of claim 3, wherein forming the training dataset further comprises removing a baseline drift from each of the single-lead ECGs.

6. The method of claim 2, wherein forming the training dataset comprises isolating a signal from at least two leads of the 12-lead ECGs, the isolated signals from the at least two leads forming a multi-lead ECG.

7. The method of claim 1, wherein the training dataset includes at least two of each ECG in the ECG dataset.

8. The method of claim 1, wherein the random gaussian noise comprises random gaussian noise isolated from one or more distinct frequency ranges.

9. The method of claim 8, wherein the random gaussian noise is isolated using at least one of high and low pass filtering.

10. The method of claim 8, wherein including the random gaussian noise with each ECG comprises randomly selecting one of the distinct frequency ranges with each of the ECGs and a random small section of the isolated noise in claim 8.

11. The method of claim 8, wherein the frequency range includes at least one of 3-12 Hz, 12-50 Hz, 50-100 Hz, or 100-150 Hz.

12. The method of claim 8, wherein including the random gaussian noise with each ECG comprises randomly selecting with each of the ECGs at least one of the distinct frequency ranges and a random small section of isolated noise from one or more of 3-12 Hz, 12-50 Hz, 50-100 Hz, or 100-150 Hz.

13. The method of claim 1, wherein the random gaussian noise is included with the ECG at a randomly chosen signal-to-noise ratio of from <1 to >1.

14. The method of claim 1, wherein the model comprises a convolutional neural network (CNN) model.

15. The method of claim 1, wherein the cardiovascular disease is left ventricular systolic dysfunction (LVSD).

16. A computer-implemented method of detecting cardiovascular disease in a subject, the method comprising:
receiving electrocardiogram (ECG) data for the subject from a portable ECG device;
applying a deep neural network to the ECG data for the subject, the deep neural network comprising a plurality of nodes trained to distinguish ECG data of a heart with cardiovascular disease from ECG data of a healthy heart according to the method of claim 1;
comparing outputs of the nodes to patterns of node outputs for ECG data from healthy subjects and subjects with one or more cardiovascular diseases; and
determining if the subject has cardiovascular disease based upon the outputs of the nodes.

17. The method of claim 16, wherein the portable ECG device is a wearable ECG device.

18. The method of claim 16, wherein the ECG data is single-lead ECG data.

19. The method of claim 18, wherein the single-lead ECG data is lead I ECG data.

20. An apparatus for detecting cardiovascular disease in a subject from portable electrocardiogram (ECG) signal data, the apparatus comprising:
a processor;
a memory unit; and
a communication interface;
wherein the processor is connected to the memory unit and the communication interface; and
wherein the processor and memory unit are configured to implement the model trained according to claim 1.
